(19) United States
(12) Patent Application Publication
(10) Pub. No.: US 2024/0344939 A1
(43) Pub. Date: Oct. 17, 2024

(54) DEGREASING COMPOSITION FOR CLEARING BIOLOGICAL TISSUE

(71) Applicant: WESTLAKE UNIVERSITY, Hangzhou (CN)

(72) Inventors: Liang Gao, Zhejiang (CN); Ruili Feng, Hangzhou (CN); Yanlu Chen, Hangzhou (CN)

(21) Appl. No.: 18/294,470

(22) PCT Filed: Mar. 9, 2022
(86) PCT No.: PCT/CN2022/079852

§ 371 Date: Feb. 1, 2024

(30) Foreign Application Priority Data
Aug. 2, 2021 (CN) 202110879793.9

(51) Int. Cl. G01N 1/28

(52) U.S. Cl. CPC G01N 1/28

(57) ABSTRACT

A degreasing composition for clearing biological tissue, a use in the preparation of a degreasing reagent, a degreasing reagent, a clearing kit comprising the degreasing composition or the degreasing reagent, a biological tissue clearing treatment method using the degreasing reagent, and an imaging method for clearing biological tissue comprising the steps of the biological tissue clearing treatment method. The degreasing composition comprises urea, N-butyldiethanolamine and Triton X-100, wherein the mass ratio of urea, N-butyldiethanolamine and Triton X-100 is (0.6-5):(0.3-3):1. The degreasing composition has a fast degreasing speed, good degreasing capabilities for large-volume biological samples, and may retain the structural features of biological tissues well from the cellular level to the subcellular level.

### TECHNICAL FIELD

The present invention relates to the technical field of biological tissue clearing, and specifically relates to a delipidation composition, use of the delipidation composition for preparing a delipidation reagent, a delipidation reagent, a clearing kit containing the delipidation composition, a tissue clearing method by using the delipidation reagent, and a biological tissue clearing and imaging method comprising the steps of the biological tissue clearing method.

### BACKGROUND

High-resolution three-dimensional fluorescence imaging of biological tissues is an effective means to obtain the three-dimensional structure of biological tissues and study biological issues such as gene expression, cell morphology, and cell distribution at the subcellular, cellular, and tissue level. Since biological tissues are opaque, traditional methods for high-resolution three-dimensional imaging of biological tissues require sectioning, two-dimensional imaging, and three-dimensional reconstruction of biological tissues [1-7]. However, these methods suffer low imaging efficiency, cumbersome sample pretreatment, and complicated data reconstruction. Furthermore, tissue sectioning could destroy the integrity and continuity of biological tissues. These problems make it difficult to apply the technology of biological tissue sectioning, imaging and reconstruction widely [8-12].

Biological tissue clearing technology renders biological tissues transparent, thereby overcoming the main obstacle in using fluorescence microscopy for high-resolution three-dimensional imaging of biological tissues, so that cutting-edge three-dimensional fluorescence microscopy imaging technologies such as light sheet microscopy technology can be used to efficiently obtain three-dimensional structural information of various biological tissues at cellular and subcellular levels, and thereby helping researchers better understand the structure and function of biological tissues and organs [13-16]. Due to this significant advantage, biological tissue clearing technology has been quickly applied to various fields of life science research.

Biological tissue clearing technologies may be roughly divided into three categories: hydrophobic [12-21], hydrophilic [22-27], and hydrogel-based [28-38] methods. Different clearing methods have different performances and show different effects on the three-dimensional imaging results of cleared biological samples.

In a hydrophobic method, such as uDISCO, a hydrophobic organic solvent is used to perform dehydration, delipidation and refractive index matching on biological tissues [18]. Hydrophobic methods usually have strong clearing abilities for biological tissues, but are more destructive for endogenous fluorescent proteins in the sample. In addition, the refractive index of samples treated by hydrophobic clearing methods is about 1.55. There are few high numerical aperture objective lenses that could match such a high refractive index to perform high-resolution imaging of the cleared sample, thus hinders the high-resolution three-dimensional imaging of biological samples treated by hydrophobic clearing methods.

In a hydrogel-based clearing method, such as CLARITY, a monomer such as acrylamide is used to form a hydrogel [27]. Most of the amino-containing protein molecules are fixed on the hydrogel structure through cross-linking, and then the sample is delipidated and cleared through electrophoresis or a detergent such as sodium dodecyl sulfate (SDS). The final refractive index of the sample is about 1.33, which is within the refractive index matching range of most high numerical aperture objective lenses. However, the tissue clearing procedure using hydrogel-based clearing methods are usually more complicated. The prepared sample could be easily damaged during the preparation. It also causes significant loss of the endogenous fluorescent proteins in the sample, which greatly limits the use of hydrogel-based methods.

In hydrophilic methods, such as CUBIC, a hydrophilic chemical reagent is used to perform delipidation and refractive index matching on biological tissues [22-24]. Hydrophilic methods have high biocompatibility and biosafety, significant advantages in retaining endogenous fluorescent proteins, and are compatible with immunostaining. Therefore, hydrophilic methods are suitable for tissue clearing of biological tissue samples labeled either with endogenous fluorescent proteins or through immunostaining. The final refractive index of the sample cleared by hydrophilic methods is about 1.49, which is within the matching range of the refractive index of most high numerical aperture microscope objective lenses, which is more appropriate for high-resolution imaging.

However, the tissue clearing capabilities of hydrophilic methods are limited, and the tissue clearing speed is usually slow. Therefore, the obtained tissue transparency using hydrophilic methods on large-volume biological samples might not be ideal. For example, the CUBIC-L agent, which is among the fastest hydrophilic methods, still needs about 10 days to clear a whole adult mouse brain completely [23].

Therefore, more efficient clearing reagents with better clearing effect are still desired.

### SUMMARY OF THE INVENTION

The inventor has developed a delipidation composition based on the existing CUBIC method. Compared with existing delipidation reagent, the clearing method using the new invented delipidation composition has a better delipidation efficiency and a stronger clearing ability for large-volume biological samples. The new method also effectively maintains the structural features of biological tissues from cellular to subcellular levels.

An objective of the present invention is to provide a delipidation composition.

Another objective of the present invention is to provide a use of the delipidation composition for preparing a delipidation reagent.

Another objective of the present invention is to provide a delipidation reagent.

Another objective of the present invention is to provide a kit for clearing biological tissues.

Another objective of the present invention is to provide a method for clearing biological tissues.

Another objective of the present invention is to provide a biological tissue clearing and imaging method.

In one aspect, the present invention provides a delipidation composition comprising urea, N-butyldiethanolamine and Triton X-100, wherein the mass ratio of urea, N-butyldiethanolamine and Triton X-100 is approximately (0.6-5):(0.3-3): 1.

The three ingredients in the delipidation composition of the present invention may be stored separately and mixed together at use, or they may be mixed together as a single product.

In the delipidation composition of the present invention, the mass ratio of urea, N-butyldiethanolamine and Triton X-100 is, but not limited to, (0.6-5):(0.3-3): 1, preferably (1-2.25):(0.6-1.5): 1, for example, it may be 1:0.6:1, 1:0.8:1, 1:1:1, 1.2:1:1, 1.5:1:1, 2:1.5:1, etc., wherein, 1.5:1:1 is the most preferred. When the ratio is within the above range, the delipidation performance of fast delipidataion speed and effective cell morphology preservation could be achieved. When the ratio is beyond the above range, for example, if the ratio of urea is too high, the tissue would be easily broken, and if the ratio of urea is too low, the delipidation speed would be slow; if the ratio of N-butyldiethanolamine is too high, the tissue would be easily broken, and if the ratio of N-butyldiethanolamine is too low, the delipidation speed would be slow; if the ratio of Triton X-100 is too high, the cell membrane would be seriously damaged, and if the ratio of Triton X-100 is too low, the delipidation speed would be slow.

The delipidation composition of the present invention may also contain a solvent, such as water. In the case that water is contained, the three ingredients in the delipidation composition of the present invention may be formulated separately into a solution and then mixed together at use, or they may also be mixed together to form a single solution.

In addition, as needed, the delipidation composition of the present invention may also include other ingredient, which may be one or more selected from the group consisting of disodium ethylenediaminetetraacetate, N,N,N′,N′-tetrakis(2-hydroxypropyl)ethylenediamine, for example, N,N,N′,N′-tetrakis(2-hydroxypropyl)ethylenediamine may be added for decolorization. The amount of the other ingredient is within the scope of those skilled in the art. For example, considering the use purpose and the desired effect, an appropriate amount may be determined by conventional methods.

The inventor found that the delipidation time may be greatly shortened by formulating a delipidation reagent using the above delipidation composition and then delipidating a biological tissue (such as the whole brain or spinal cord of an adult mice).

Accordingly, in another aspect, the present invention provides a use of the invented delipidation composition in preparation of a delipidation reagent.

In another aspect, the present invention provides a delipidation reagent, which is an aqueous solution containing, by mass percentage concentration, 10 to 25% of urea, 5 to 15% of N-butyldiethanolamine and 5 to 15% of Triton X-100.

In the delipidation reagent of the present invention, the mass percentage concentration of urea is 10 to 25%, preferably 12 to 18%, such as 12.5%, 13%, 13.5%, 14%, 14.5%, 15%, 15.5%, 16%, 16.5%, 17%, 18%, etc., more preferably 15%. Within the above range, the delipidation performance, such as high delipidation speed, good cell morphology maintenance, and endogenous fluorescent protein preservation, could be achieved. If the urea content is too high, the delipidation speed could be faster, but the tissue would be easily damaged, whereas if the urea ratio is too low, the delipidation speed would be slower, and the endogenous fluorescent protein would be easily quenched.

In the delipidation reagent of the present invention, the mass percentage concentration of N-butyldiethanolamine is 5 to 15%, preferably 8 to 12%, such as 8.5%, 9%, 9.5%, 10%, 10.5%, 11%, 11.5%, etc., more preferably 10%. Within the above range, the delipidation performance, such as fast delipidation speed, good retention of endogenous fluorescent proteins and good cell morphology preservation, could be achieved. If the ratio of N-butyldiethanolamine is too high, the delipidation speed would be faster, but the tissue would be easily damaged, whereas if the ratio of N-butyldiethanolamine is too low, the delipidation speed would be slow and the endogenous fluorescent protein retention would be poor.

In the delipidation reagent of the present invention, the mass percentage concentration of Triton X-100 is 5 to 15%, preferably 8 to 12%, such as 8.5%, 9%, 9.5%, 10%, 10.5%, 11%, 11.5%, etc., more preferably 10%. Within the above range, the delipidation performance, such as fast delipidation speed and good cell morphology preservation could be achieved. If the ratio of Triton X-100 is too high, the cell morphology would be severely damaged, whereas if the ratio of Triton X-100 is too low, the delipidation speed would be slow.

In addition, if needed, the delipidation reagent of the present invention may also include other ingredients, and the ingredients may be one or more selected from the group consisting of disodium ethylenediaminetetraacetate and N,N,N′,N′-tetrakis(2-hydroxypropyl)ethylenediamine. For example, N,N,N′,N′-tetrakis(2-hydroxypropyl)ethylenediamine may be added for decolorization. The amount of the additionally added ingredient is within the scope of those skilled in the art. For example, considering the use purpose and the desired final effect, an appropriate amount may be determined by conventional methods.

The preparation method of the delipidation reagent according to the present invention is not particularly limited. For example, the respective ingredients of the delipidation composition may be dissolved in pure water respectively, and then mixed together and diluted to volume, or the respective ingredients of the delipidation composition may be dissolved simultaneously in a specified amount of pure water.

In another aspect, the present invention provides a kit for biological tissue clearing and imaging, which comprises the above-mentioned delipidation composition or the above-mentioned delipidation reagent.

The kit may also include one or more selected from the group consisting of a phosphate buffer (PB), paraformaldehyde (PFA), a refractive index matching composition, a gelling reagent, an imaging buffer, a staining reagent, etc. In addition, the kit may also include an instruction providing information of the relevant reagents and instructing how to formulate the relevant reagents. The instruction may be recorded on an appropriate medium, such as paper, or may be stored in an appropriate storage medium, such as film, magnetic disk, optical disk, USB disk, hard disk, etc. Alternatively, the kit may include a medium recording an indication on how to obtain the instruction, such as paper printed with a two dimensional code or URL link information of the instruction, a film carrying a two dimensional code or URL link information of the instruction, or a disk, optical disk, USB flash drive, hard disk, etc. stored with a two dimensional code image or URL link information of the instruction, wherein the instruction may be downloaded or a website where the instruction may be downloaded may be linked to by scanning the two dimensional code.

The phosphate buffer may be a phosphate buffer suitable for processing biological tissues. For example, it may be a phosphate buffer composed of disodium hydrogen phosphate and sodium dihydrogen phosphate with a pH of 7.0 to 7.4, such as 7.1, 7.15, 7.2, 7.25, 7.3, 7.35, etc., preferably a phosphate buffer with a pH value of about 7.2. Those skilled in the art may appropriately select the amounts of disodium hydrogen phosphate and sodium dihydrogen phosphate according to the final pH required. In the kit, the phosphate buffer may be a solid composed of disodium hydrogen phosphate and sodium dihydrogen phosphate, which may be prepared into a suitable solution at use, or may be a phosphate buffer solution prepared from disodium hydrogen phosphate and sodium dihydrogen phosphate. In one embodiment, a 0.1 M phosphate buffer with a pH of 7.2, which is prepared from disodium hydrogen phosphate and sodium dihydrogen phosphate, may be used. Here, the concentration of the phosphate buffer solution is calculated as the concentration of the phosphate radical.

Paraformaldehyde is used to prepare a fixative solution for processing biological tissues, and the fixative solution is used to fix the biological tissues. In the kit, it may be a solid, and at use, it may be prepared as a solution of paraformaldehyde in a phosphate buffer with a mass volume concentration of 4%, or it may be a solution. In one embodiment, the paraformaldehyde is a 4% paraformaldehyde solution (mass (g)/volume (ml) concentration) with a pH of 7.2-7.4, which is obtained by dissolving paraformaldehyde in the above-mentioned phosphate buffer (e.g. 0.1 M) and adjusting the pH.

The refractive index matching composition may be a composition of any formulation suitable for preparing a refractive index matching solution in the CUBIC method. The refractive index of the prepared refractive index matching solution may be 1.46 to 1.50 to meet the imaging requirements of optical microscopes. In the kit, the refractive index matching composition may be a solid composed of refractive index matching agents in a certain formulation, which is prepared into a suitable refractive index matching solution at use; or the refractive index matching composition may be a prepared refractive index matching solution. That is, the refractive index matching composition may be a solid composed of refractive index matching agents from which the prepared refractive index matching solution has a refractive index of 1.46 to 1.50, or a refractive index matching solution with a refractive index of 1.46 to 1.50.

In some embodiments, the refractive index matching composition includes urea, sucrose, antipyrine and trihydroxyethylamine, wherein the ratio of urea, sucrose, antipyrine and trihydroxyethylamine is (1-7):(1-6.5):(1-6.5): 1, especially (1.6-3.8):(1.6-3.2):(1.6-3.2): 1, such as 1.7:1.7:1.7:1, 2:1.8:1.9:1, 2.5:2:2:1, 2.5:2.25:2.25:1, 3:2:2:1, 3:2.5:2.5:1, 3.5:3:2.5:1, 3.5:3:3:1, etc., more specifically about 2.5:2.25:2.25:1.

In some embodiments, the refractive index matching composition is an aqueous solution, which contains, by mass percentage concentration, 15 to 35%, preferably 20 to 30%, such as 21%, 22%, 23%, 24%, 25%, 26%, 27%, 28%, 29%, etc., especially 25% of urea, 15 to 32.5%, preferably 20 to 25%, such as 21%, 21.5%, 22%, 22.5%, 23%, 23.5%, 24%, 24.5%, etc., especially 22.5% of sucrose, 15 to 32.5%, preferably 20 to 25%, such as 21%, 21.5%, 22%, 22.5%, 23%, 23.5%, 24%, 24.5%, etc., especially 22.5% of antipyrine, and 5 to 15%, preferably 8 to 12%, such as 9%, 9.5%, 10%, 10.5%, 11%, 11.5%, etc., especially 10% of trihydroxyethylamine.

The gelling reagent may be agarose, and is used to prepare a gelling solution, which is prepared by dissolving agarose in a refractive index matching solution. In the kit, the gelling reagent may be an agarose solid to be prepared into a suitable solution at use, or a prepared gelling solution. In some embodiments, the gelling solution is an agarose solution of 1.5% to 3%, preferably 1.8% to 2.5%, such as 1.85%, 1.90%, 1.95%, 2.0%, 2.05%, 2.10%, 2.15%, 2.20%, 2.25%, 2.30%, 2.35%, 2.40%, 2.45%, etc., especially about 2%, by mass percentage concentration.

The imaging buffer is a mixture of silicone oil and mineral oil. It has a refractive index nearly the same as that of the refractive index matching solution. Its refractive index may be 99.8% to 100.06%, preferably 99.9% to 100.01% of the refractive index of the refractive index matching solution. In some embodiments, the imaging buffer is a mixture of silicone oil and mineral oil with a refractive index of 1.494. There is no particular restriction on the preparation method of the imaging buffer. For example, it may be prepared by adding mineral oil to silicone oil while measuring the refractive index until the final refractive index reaches the target value.

In some embodiments, the kit includes:
1. a fast delipidation solution (S1), which is an aqueous solution containing, by mass percentage concentration, 15% of urea, 10% of N-butyldiethanolamine and 10% of Triton X-100;
2. a refractive index (RI) matching solution (S2), which is an aqueous solution containing, by mass percentage concentration, 25% of urea, 22.5% of sucrose, 22.5% of antipyrine and 10% of trihydroxyethylamine;
3. a gelling solution (S3), which is a solution of 2% by mass percentage concentration of agarose in a refractive index matching solution;
4. an imaging buffer (S4), which is a mixture of silicone oil and mineral oil with a refractive index of 1.494.

In another aspect, the present invention provides a method for clearing a biological tissue, comprising: delipidating the biological tissue sample in the delipidation reagent according to the present invention until the sample is transparent, and then matching the refractive index of the biological tissue sample in a refractive index matching solution.

In some embodiments, in the method for clearing a biological tissue, the clearing may be performed under shaking (for example, on a shaker).

In some embodiments, in the method for clearing a biological tissue, the delipidation reagent according to the present invention may be replaced regularly or irregularly, for example, every 6 hours, every 12 hours, or every 24 hours, but the present invention is not limited thereto. In the method for clearing a biological tissue, the amount of the delipidation reagent according to the present invention at single use is not particularly limited, as long as it immerses the biological tissue sample. In particular, the specific amount may be determined based on the volume of the biological tissue sample. For example, the amount of the delipidation reagent at a single use may be 5 to 25 times, preferably 10 to 20 times, especially 15 times of the volume of the sample.

In some embodiments, in the method for clearing a biological tissue, the delipidation time may be 5 minutes or more, 1 hour or more, 1 day or more, etc., but is not limited thereto. The specific delipidation time may be varied according to the volume and age of the biological tissue, the amount of the delipidation reagent, etc. For example, by using the delipidation solution of the present invention, the delipidation may be achieved in about 10 minutes for a 200 μm thick mouse brain slice; the delipidation may be achieved in about 2 to 3 days for the whole brain and spinal cord of an adult mouse; and the delipidation may be achieved in about 7 days for a whole P7 rabbit brain.

In the method for clearing a biological tissue, refractive index matching may be performed by immersing the delipidated biological tissue sample in a refractive index matching solution.

The refractive index matching solution is not particularly limited, and any suitable refractive index matching solution known in the art could be used, or the above-mentioned refractive index matching solution could be used without special restrictions.

In particular, the refractive index matching solution is an aqueous solution containing, by mass percentage concentration, 15 to 35% preferably 20 to 30%, such as 21%, 22%, 23%, 24%, 25%, 26%, 27%, 28%, 29%, etc., especially 25% of urea, 15 to 32.5%, preferably 20 to 25%, such as 21%, 21.5%, 22%, 22.5%, 23%, 23.5%, 24%, 24.5%, etc., especially 22.5% of sucrose, 15 to 32.5%, preferably 20 to 25%, such as 21%, 21.5%, 22%, 22.5%, 23%, 23.5%, 24%, 24.5%, etc., especially 22.5% of antipyrine, and 5 to 15%, preferably 8 to 12%, such as 9%, 9.5%, 10%, 10.5%, 11%, 11.5%, etc., especially 10% of trihydroxyethylamine.

In some embodiments, the refractive index matching may be performed under shaking (eg, on a shaker).

In some embodiments, during the refractive index matching, the refractive index matching solution may be replaced regularly or irregularly, for example, every 6 hours, every 12 hours, or every 24 hours, but the present invention is not limited thereto. The amount of the refractive index matching solution at a single use is not particularly limited as long as it immerses the biological tissue sample. In particular, the specific amount may be determined based on the volume of the biological tissue sample, and the amount of the refractive index matching solution at a single use may be 2 to 22 times, preferably 7 to 17 times, especially 12 times of the volume of the sample.

In some embodiments, during the above refractive index matching, the immersing time using the refractive index matching solution may be 5 minutes or more, 1 hour or more, 1 day or more, etc., but is not limited thereto. The specific immersing time may be varied according to the volume and age of the biological tissue, the amount of the refractive index matching solution, etc. For example, the immersing may last for about 2 to 3 days for the whole brain and spinal cord of an adult mouse; the immersing may last for about 7 days for the whole brain of a P7 rabbit.

In another aspect, the present invention provides a biological tissue clearing and imaging method comprising a step of clearing the biological tissue sample using the method for clearing a biological tissue according to the present invention.

The imaging method could also include other steps required for imaging, such as collecting and fixing a biological tissue before clearing, and gel embedding, imaging, or staining, sectioning and imaging, etc., after clearing, but the invention is not limited thereto.

In one embodiment, the imaging method comprises the steps of:
1. fixing a biological tissue sample;
2. clearing the fixed biological tissue sample with the method for clearing a biological tissue according to the present invention;
3. gel-embedding the cleared biological tissue sample;
4. imaging the gel-embedded biological tissue sample.

In the above step 1, there is no particular limitation on the fixation of the biological tissue sample, and any conventional method in the art may be used. For example, a biological tissue isolated from an organism may be immersed overnight in a paraformaldehyde solution (e.g, a 4% (g/mL) solution of paraformaldehyde in a 0.1 M phosphate buffer with a pH of 7.2-7.4) to complete the fixing. However, the present invention is not limited thereto.

In the above step 2, the description about the clearing step is the same as the description of the above-mentioned method for clearing a biological tissue according to the present invention, and will not be repeated here.

In the above step 3, the gel embedding refers to embedding the refractive index-matched biological tissue sample in gel. There is no particular limitation on the method of embedding the biological tissue sample in gel, and any known suitable method may be used. For example, it may be performed by adding a gelling solution into a mold, and then adding the biological tissue sample, and then gelling at the same time to obtain a gel-embedded biological tissue sample.

The gelling solution may be prepared by dissolving any suitable gelling reagent in a refractive index matching solution. In some embodiments, the gelling reagent is agarose. In some embodiments, the gelling solution is a solution of 1.5% to 3%, preferably 1.8% to 2.5%, such as 1.85%, 1.90%, 1.95%, 2.0%, 2.05%, 2.10%, 2.15%, 2.20%, 2.25%, 2.30%, 2.35%, 2.40%, 2.45%, etc., especially about 2% by mass percentage concentration of agarose in a refractive index matching solution.

Gelling may be achieved by dissolving the gelling reagent in a refractive index matching solution at a high temperature and then cooling; or by adding a cross-linking agent, but the invention is not limited thereto.

In the above step 4, imaging may be performed according to any imaging method known in the art. For example, it may be performed by mounting a gel-embedded biological tissue sample on a sample holder of an imaging microscope. When imaging, the biological tissue sample needs to be immersed in an imaging buffer.

The imaging buffer protects the sample from shrinkage due to evaporation and dehydration and prevents excitation light from multiple refractions before entering into the sample to affect the imaging quality of the imaging. Its refractive index is nearly the same as that of the refractive index matching solution, which may be 99.8% to 100.06%, preferably 99.9% to 100.01% of the refractive index of the refractive index matching solution.

In some embodiments, the imaging buffer is a mixture of silicone oil and mineral oil. In particular, the imaging buffer is a mixture of silicone oil and mineral oil with a refractive index of 1.494.

In this description, all characteristics or conditions defined in the form of numerical ranges or percentage ranges are for brevity and convenience only. Accordingly, descriptions of numerical ranges or percentage ranges shall be deemed to cover and specifically disclose all possible subranges and individual values within the ranges, in particular integer values. For example, a range of “1 to 8” should be deemed to have specifically disclosed all sub-ranges such as 1 to 7, 2 to 8, 2 to 6, 3 to 6, 4 to 8, 3 to 8, etc., particularly, a secondary range defined by all integer values and shall be deemed to have been specifically disclosed as individual values within the range such as 1, 2, 3, 4, 5, 6, 7, 8, etc. Unless otherwise indicated, the foregoing method of interpretation applies to all contents of the present invention, regardless of the scope is broad or not.

If a quantity or other numerical value or parameter is expressed as a range, a preferred range, or a series of upper and lower limits, it should be understood that all the ranges consisted of any pairing of the upper or preferred value of the range and the lower or preferred value of the range have been specifically disclosed herein, regardless of these ranges are separately disclosed or not. In addition, when a value range is mentioned herein, such range shall include its endpoints and all integers and fractions within the range, unless otherwise stated.

In the description, a numerical value shall be understood to have an accuracy of the significant digits of the numerical value, provided that the object of the invention is achieved. For example, the number 40.0 should be understood to cover the range from 39.50 to 40.49. With the exception of the working examples provided at the end of the detailed description, all numerical values for parameters (e.g. quantities or conditions) in this specification (including the appended claims) are to be understood in all cases as being modified by the term “about”, regardless of whether “about” actually appears before the value. “About” means that the stated value is allowed to be slightly less precise (somewhere near exact at the value; about or reasonably close to the value; approximately). If the imprecision provided by “about” is not understood in this ordinary sense in the art, “about” as used herein at least means the variation that may be produced by ordinary methods of measuring and using these parameters. For example, “about” may include a variation of less than or equal to 10%, less than or equal to 5%, less than or equal to 4%, less than or equal to 3%, less than or equal to 2%, less than or equal to 1%, or less than or equal to 0.5%, and in some respects, a variation of less than or equal to 0.1%.

The present invention has been described in details above, but the above-mentioned embodiments are only illustrative in nature and are not intended to limit the present invention. Furthermore, the description is not limited by any theory presented in the preceding prior art or summary of the invention or described in the following examples.

### Beneficial Effects

The present invention provides a delipidation reagent with high clearing efficiency and strong clearing ability for biological samples. Compared with the delipidation reagents used in existing hydrophilic clearing methods, the delipidation reagent not only has the advantages of high biocompatibility, high biosafety, and good retention of endogenous fluorescent proteins of general delipidation reagent, but also has the following advantages over the existing delipidation reagents:

First, the delipidation reagent according to the present invention has a faster clearing speed with better clearing ability for large-volume biological samples;

Secondly, during the clearing process a biological sample with the delipidation reagent according to the present invention, the deformation of biological samples caused by delipidation is smoother, thereby the integrity of biological tissues can be better maintained.

Thirdly, the delipidation reagent according to the present invention may well maintain the structural characteristics of biological tissues at cellular to subcellular levels.

### BRIEF DESCRIPTION OF DRAWINGS

FIG. 1 is a picture showing the clearing process of a whole mouse brain using the delipidation solution, refractive index matching solution and gelling solution according to the present invention, and the photos of the mouse brain after each step of the process

FIG. 2 is a picture showing the clearing process of a mouse spinal cord using the delipidation solution, refractive index matching solution and gelling solution according to the present invention, and the photos of the mouse spinal cord after each step of the process.

FIG. 3 is a picture showing the tissue clearing efficiency comparison of the present invention and CUBIC-L, wherein A shows the transparency changing processes of whole adult mouse brains cleared using the delipidation solution of the present invention and CUBIC-L respectively. B shows the transparency changing processes of the whole P7 rabbit brains cleared using the delipidation solution of the present invention and CUBIC-L. The scale bars are all 1 cm.

FIG. 4 is a picture showing the comparison of morphological changes during the clearing process of mouse brain slices with the delipidation solution according to the present invention and the CUBIC and CUBIC-L delipidation solutions in the prior art, wherein A is a schematic diagram of the experimental method for preparing mouse brain slice samples and recording the morphological changes during the clearing process of mouse brain slices. B-D show the morphological changes of mouse brain slices during the clearing process, wherein B shows the morphological changes of mouse brain slices during their clearing process with the prior art CUBIC and CUBIC-L delipidation solutions and the delipidation solution S1 according to the present invention, the scale bar is 5 mm; C shows the area change percentage of mouse brain slices; D shows the average area change percentage after the clearing of mouse brain slices reached equilibrium.

FIG. 5 shows the changes in cell distribution and morphology of Thy1-GFP-M mouse brain slices before and after treatment with the delipidation solution S1 of the present invention, wherein, A shows the changes in cell distribution in the amygdala area before and after the clearing process, and the scale bar is 100 μm; B shows the changes in cell morphology of a single neuron cell in the cortex area before and after the clearing process. The scale bars are (a, d) 30 μm, (b, e) 5 μm, (c, f) 0.5 μm.

FIG. 6 shows the three-dimensional imaging of a whole mouse brain cleared with the delipidation solution S1 of the present invention at spatial resolutions of micron to sub-micron levels, wherein A and B are lateral and axial projection views of the three-dimensional imaging results of the cleared whole brain of a Thy1-eGFP mouse using a 0.25 numerical aperture (NA) air detection objective lens at a spatial resolution of 2×2×5 μm³, the scale bar is 1 mm; C is the axis projection view of the sample area marked in A, the scale bar is 200 μm; D is the axial projection view of the three-dimensional imaging results obtained by imaging the same area in C using a 0.5 NA immersion detection objective lens with a refractive index matched to 1.49 at a spatial resolution of 0.6×0.6×1.5 μm³, the scale bar is 200 μm; E-H are lateral and axial projection views of the two 100×100×100 μm³ areas marked in D, the scale bar is 10 μm.

### DETAILED EMBODIMENTS

Hereinafter, preferred examples are provided for those skilled in the art to better understand the present invention. However, the following examples are provided only for understanding the present invention easier, and the scope of the present invention is not limited thereto. Furthermore, the present invention is not bounded by any theory described in the preceding prior art or summary of the invention or the following examples. Unless otherwise stated, the methods, reagents and conditions used in the examples are conventional methods, reagents and conditions in the art.

### EXAMPLES

### 1. Reagents and Instruments

Sodium hydrogen phosphate dodecahydrate (Na₂HPO₄·12H₂O) and sodium dihydrogenphosphate dihydrate (NaH₂PO₄·2H₂O), were purchased from Sangon Biotech (Shanghai) Co. Ltd.

Paraformaldehyde, was purchased from Sinopharm Chemical Reagent Co., Ltd., product No.: 80096618.

Urea was purchased from Sangon Biotech (Shanghai) Co. Ltd., product No.: A600148-9001

Triton X-100 was purchased from Sangon Biotech (Shanghai) Co. Ltd., product No.: A110694-9001

N-butyldiethanolamine was purchased from TCI, product No.: B0725

Sucrose was purchased from Sangon Biotech (Shanghai) Co. Ltd., product No.: A610498-0005

Antipyrine was purchased from Sigma-Aldrich, product No.: V900710-500G

Triethanolamine was purchased from Sigma-Aldrich, product No.: V900257-500ML Agarose S was purchased from Nacalai Tesque, product No.: 01163-76-500G

Mineral oil was purchased from Sigma-Aldrich, product No.: M8410-1L

Silicone oil AP 100 was purchased from Sigma-Aldrich, product No.: 10838-500ML

Imaging was performed on a stereomicroscope (OLYMPUS SZ61), a fluorescence stereomicroscope (ZEISS Axio Zoom.V16), a laser confocal microscope (ZEISS LSM800), and a light sheet microscope self-built in laboratory for large sample imaging.

### 2. Solution Preparation

### (1) 0.1M Phosphate Buffer (PB)

28.998 g of Na₂HPO₄·12H₂O and 2.964 g of NaH₂PO₄·2H₂O were dissolved in 900 ml of deionized water (dd H₂O), and the resulting solution was adjusted to a pH of 7.2, and finally brought to a volume of 1 L.

### (2) 4% Paraformaldehyde (PFA)

40 g of paraformaldehyde (PFA) was added to 900 ml of 0.1M PB, and a small amount of sodium hydroxide was added thereto while stirring to promote the dissolution of PFA. When PFA was completely dissolved, the solution was adjusted to pH 7.2-7.4 by dropping concentrated saline and brought to a volume of 1 L. 4% PFA may be stored at 4° C. for 1 month, while it is recommended to prepare it fresh before use.

### (3) Fast Delipidation Solution (S1)

150 g of urea, 100 g of N-butyldiethanolamine and 100 g of Triton X-100 were dissolved in 650 g of dd H₂O, and the resultant was let stand until the bubbles therein disappeared before use.

### (4) Refractive Index (RI) Matching Solution (S2)

250 g of urea, 225 g of sucrose and 225 g of antipyrine were dissolved in 200 g of ddH₂O under heating. After the temperature returned to room temperature, 100 g of trihydroxyethylamine was added thereto and stirred evenly, and then the resultant was removed off impurities by filtering for future use.

### (5) Gelling Solution (S3)

2 g of agarose S and 98 g of S2 were mixed evenly and then the mixture was heated under microwave. After the agarose S was dissolved, the prepared gelling solution S3 was stored at 37° C.

### (6) Imaging Buffer (S4)

S4 was a mixture of silicone oil and mineral oil with a final refractive index of 1.494. S4 may be used repeatedly, and it was stirred well to eliminate bubbles before use.

### Example 1 Clearing and Imaging of a Whole Mouse Brain

### 1. Material Collection and Fixation (1 Day)

After being deeply anesthetized by intraperitoneal injection of 2% sodium pentobarbital, the mouse was perfused cardiacally with 50 ml of 0.1 M PB and 30 ml of 4% PFA pre-cooled at 4° C., and then the mouse brain was dissected. The harvested mouse brain was soaked in 40 ml of 4% PFA and placed at 4° C. overnight. After removing 4% PFA the next day, the mouse brain was washed three times with 0.01M PB for two hours each time.

### 2. Delipidation (2 to 4 Days)

The fixed mouse brain was placed in a 50 ml centrifuge tube, which was then filled with S1. The centrifuge tube was placed in a shaker at 37° C. and a shaking frequency of 60 rpm to remove greases, and S1 was replaced every 24 hours. An adult mouse brain generally needs to be delipidated for 3 days to become completely transparent. For larger samples or older biological samples, the delipidation time needs to be extended until the sample becomes transparent.

### 3. Refractive Index Matching (2 Days)

The delipidated mouse brain was placed in a 50 ml centrifuge tube, which was then filled with S2. Refractive index matching was performed in a shaker at 25° C. and a shaking frequency of 60 rpm. S2 was replaced every 24 hours.

### 4. Gel Embedding (4 Hours)

S3 was firstly added into a gel mold, and then a porous iron plate was placed at the bottom of the mold. Then the sample with matched refractive index was put into the mold. After adjusting the position of the sample to the middle of the mold, the mold was placed in a refrigerator at 4° C. for the gelling of S3, which was solidified in about 4 hours.

### 5. Imaging

The sample was taken out of the mold, mounted on the sample holder of the imaging microscope, and immersed in the imaging buffer S4 for three-dimensional imaging.

FIG. 1 schematically shows the clearing process and the photos of the whole mouse brain taken after each step.

### Example 2 Clearing and Imaging of Mouse Spinal Cord

### 1. Material Collection and Fixation (1 Day)

After being deeply anesthetized by intraperitoneal injection of 2% sodium pentobarbital, the mouse was perfused cardiacally with 50 ml of 0.1 M PB and 30 ml of 4% PFA pre-cooled at 4° C. The spinal cord was dissected from the spine and then fixed between an orifice plate and a 100-mesh nylon mesh (to ensure that the spinal cord would not bend during post-fixation and the PFA could fully reach the sample), then the sample was placed in a 50 ml centrifuge tube containing 4% PFA and fixed overnight in a shaker at 4° C. After removing 4% PFA the next day, the sample was washed with 0.01M PB three times for two hours each time to completely remove PFA.

### 2. Delipidation (2 to 3 Days)

After PFA was washed off, the centrifuge tube was filled with S1, and placed in a shaker at 37° C. at 60 rpm to remove greases, and S1 was replaced every 24 hours. The delipidation lasted for 2 to 3 days until the spinal cord was completely transparent.

### 3. Refractive Index Matching (2 Days)

The S1 agent in the centrifuge tube was replaced with S2, and refractive index matching was performed in a shaker at 25° C. and a shaking frequency of 60 rpm. S2 was replaced every 24 hours.

### 4. Gel Embedding (4 Hours)

S3 was firstly added into a spinal cord gel mold, and then a porous iron plate was placed at the bottom of the mold. Then the sample with matched refractive index was put into the mold. After adjusting the position of the sample to the middle of the mold, the mold was placed in a refrigerator at 4° C. for the gelling of S3, which was solidified in about 4 hours.

### 5. Imaging

The sample was taken out of the mold, mounted on the sample holder of an imaging microscope, and immersed in the imaging buffer S4 for three-dimensional imaging.

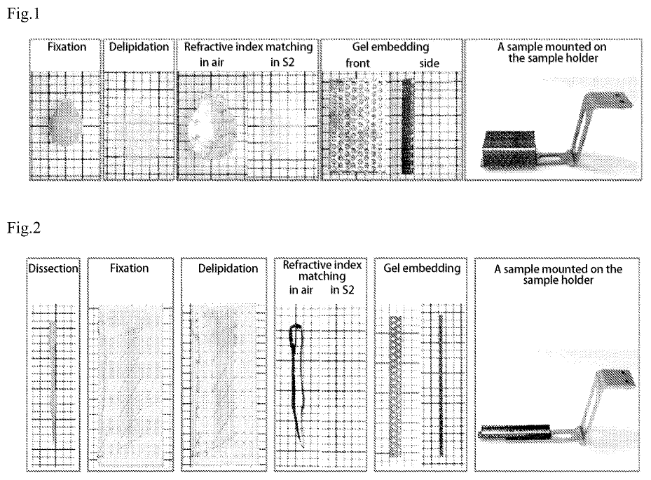

FIG. 2 schematically shows the clearing process and the photos of a whole mouse spinal cord taken after each step.

### Example 3 Clearing and Imaging of a Whole P7 Rabbit Brain

### 1. Material Collection and Fixation (1 Day)

After being deeply anesthetized by intraperitoneal injection of 2% sodium pentobarbital, a rabbit was perfused cardiacally with 100 ml of pre-cooled 0.1 M PB and 60 ml of 4% PFA, and then the rabbit brain was dissected. The rabbit brain was soaked into a blue-capped bottle containing 80 ml of 4% PFA, placed in a shaker at 4° C., and shaken overnight. After removing 4% PFA the next day, the rabbit brain was washed three times with 0.01M PB by shaking in a shaker for two hours each time.

### 2. Delipidation (2 to 4 Days)

After the PFA was washed off, the 100 ml blue cap bottle was filled with S1 for delipidation in a shaker at 37° C. and a shaking frequency of 60 rpm. S1 was replaced every 24 hours. The delipidation lasted for 2 to 4 days until the rabbit brain was completely transparent.

### 3. Refractive Index Matching (2 Days)

The S1 agent in the blue cap bottle was replaced with S2, and refractive index matching was performed in a shaker at 25° C. and a shaking frequency of 60 rpm. S2 was replaced once after 24 hours.

### 4. Gel Embedding (4 Hours)

S3 was firstly added into a rabbit brain gel mold, and then a porous iron plate was placed at the bottom of the mold. Finally, the sample with matched refractive index was put into the mold. After adjusting the position of the sample to the middle of the mold, the mold was put in a refrigerator at 4° C. for the gelling of S3, which was solidified in about 4 hours.

### Comparative Example 1 Clearing and Imaging of a Whole Mouse Brain Using CUBIC-L

A CUBIC-L delipidation solution and a refractive index matching solution were prepared as follows based on reference [27]. CUBIC-L delipidation solution was prepared as follows: 150 g of N-butyldiethanolamine and 100 g of Triton X-100 were dissolved in 850 g of ddH₂O, and the resultant was let stand until the bubbles therein disappeared before use.

The CUBIC-L refractive index matching solution was prepared as follows: 450 g of antipyrine and 300 g of nicotinamide were dissolved in 200 g of ddH₂O, and the resultant was adjusted to pH 8-9 with N-butyldiethanolamine, and then added with ddH₂O to a total mass of 1000 g. A whole mouse brain was cleared in the same manner as that in Example 1, except that the CUBIC-L delipidation solution was used for delipidation in step 2 and the CUBIC-L refractive index matching solution was used for refractive index matching in step 3.

### Comparative Example 2 Clearing and Imaging of a Whole P7 Rabbit Brain Using CUBIC-L

A whole brain of a P7 rabbit was cleared in the same manner as that in Example 3, except that the CUBIC-L delipidation solution was used for delipidation in step 2 and the CUBIC-L refractive index matching solution was used for refractive index matching in step 3.

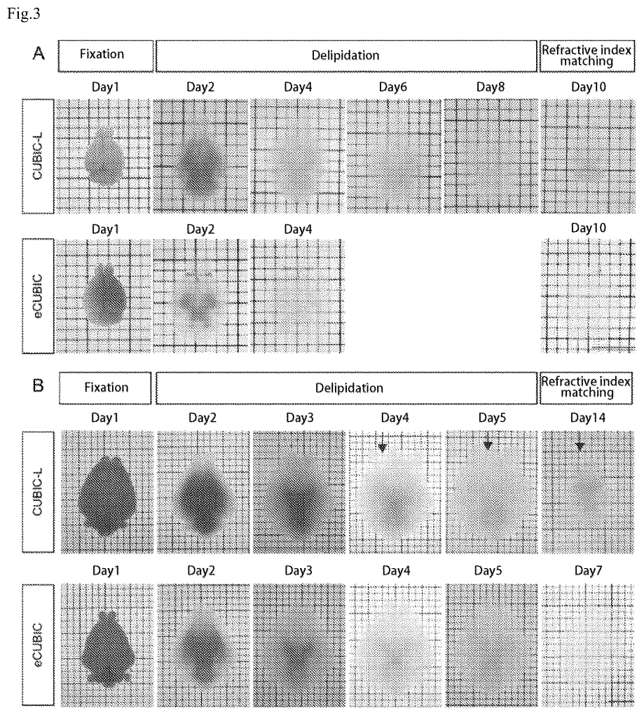

In Examples 1 and 3 and Comparative Examples 1 and 2, a photo of the sample was taken daily with a stereomicroscope (OLYMPUS SZ61) during delipidation to inspect the transparency change of the sample during clearing. The results are shown in FIG. 3.

FIG. 3 shows the tissue clearing efficiency comparison of the delipidation solution of the present invention and CUBIC-L, wherein A shows the transparency changing processes of adult mouse brains cleared using the delipidation solution of the present invention and CUBIC-L respectively, B shows the transparency changing processes of P7 rabbit brains cleared using the delipidation solution of the present invention and CUBIC-L respectively. The scale bars are all 1 cm.

From A of FIG. 3: the experimental results of the clearing the whole adult mouse brain show that the transparency of the mouse brain cleared with the delipidation solution S1 of the present invention after 4 days is equivalent to the transparency of the mouse brain cleared with CUBIC-L after 8 days. The whole mouse brain has reached complete transparency after cleared with the delipidation solution S1 of the present invention for 6 days, which is better than that of the whole mouse brain cleared with the CUBIC-L delipidation solution for 10 days.

From B of FIG. 3: the comparison results show that the transparency of the whole P7 rabbit brain cleared using the delipidation solution S1 according to the present invention after 7 days is better than that of the whole P7 rabbit brain cleared using the CUBIC-L delipidation solution for 14 days.

The experimental results show that the delipidation reagent of the present invention has higher clearing efficiency and better clearing ability for large samples than that of the general hydrophilic methods.

### Example 4 and Comparison Examples 3 and 4 Clearing of 200 μm Thick Mouse Brain Slices

The CUBIC delipidation solution was prepared as follows: 250 g of urea and 312 g of 80 wt % N,N,N′,N′-tetrakis(2-hydroxypropyl)ethylenediamine were added to 288 g of dd H₂O, and completely dissolved under heating. After the temperature returned to room temperature, 150 g of Triton X-100 was added under stirring. After dissolving, the solution was let stand until the bubbles therein disappeared for future use.

After the biological tissue was cleared, the preservation of the original tissue structure has a crucial impact on the application of tissue clearing technologies. In order to better understand the clearing process of biological tissues and compare the degree of deformation of biological tissue structures caused by different clearing methods, the clearing process of mouse brain slices was studied.

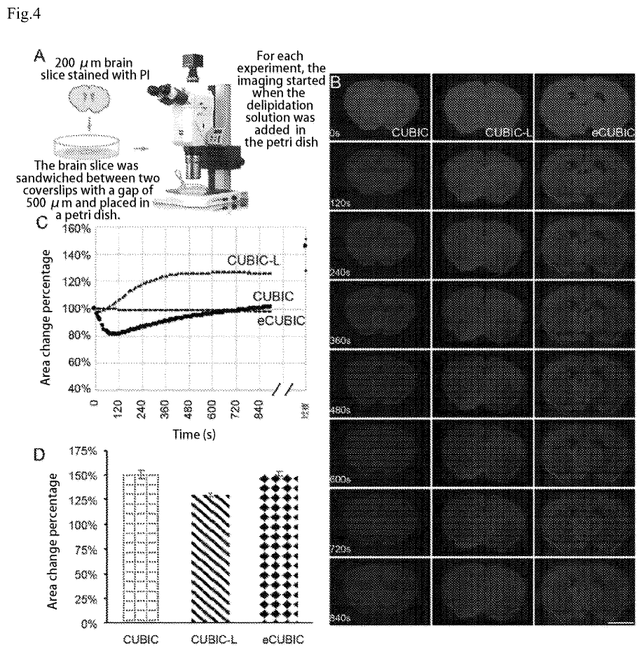

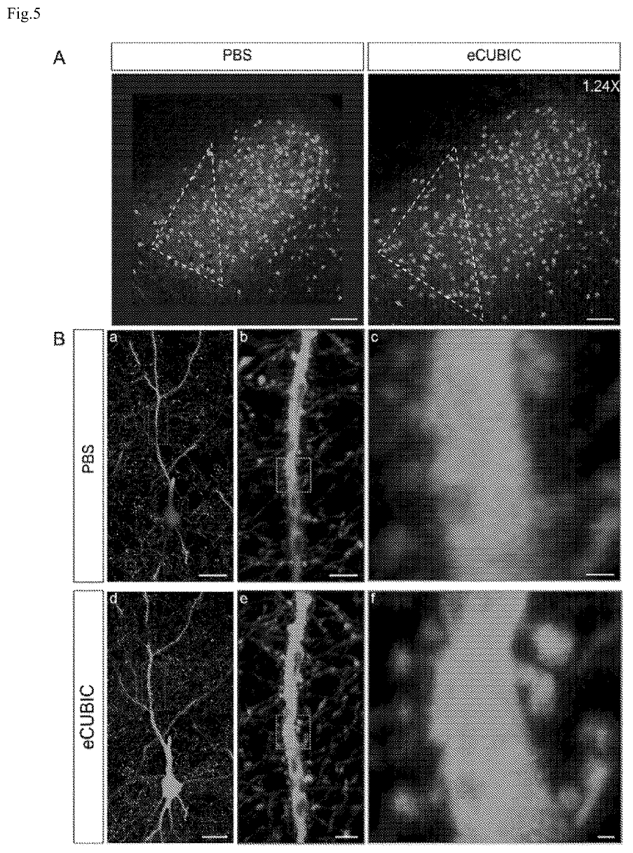

200 μm thick mouse brain slices were cleared using the delipidation solution S1 according to the present invention (Example 4), the CUBIC delipidation solution (Comparison Example 3) and the CUBIC-L delipidation solution (Comparison Example 4), respectively. A of FIG. 4 is a schematic diagram of the experimental method for preparing mouse brain slice samples and recording the deformation during the clearing processes of the mouse brain slices. The specific operation steps were as follows.

Several 200 μm thick mouse brain slices were cut from the same brain regions of a fixed wild-type adult mouse brain and stained with propidium iodide (PI). Each stained mouse brain slice was placed between two coverslips with a gap of 500 μm. The clamped brain slices were placed in different petri dishes, and the clearing reagents of the selected clearing method were added to the petri dishes respectively, and the brain slices were cleared at 23° C. The brain slices were imaged using a Zeiss V16 microscope with a photo taken every 10 seconds to record the clearing process of all brain slices and the area changes of the brain slices until the brain slices became transparent. After all brain slices were completely transparent, they were left in the corresponding clearing solution overnight to allow the deformation of the transparent brain slices to reach the final equilibrium state.

B-D of FIG. 4 show the morphological changes of the mouse brain slices during the clearing process, wherein B shows the morphological changes of mouse brain slices during clearing with the prior art CUBIC and CUBIC-L delipiation solutions and the delipidation solution S1 according to the present invention, the scale bar is 5 mm; C shows the area change percentage of mouse brain slices; D shows the average area change percentage after the clearing of mouse brain slices reached equilibrium.

Since the thickness of the brain slices was only 200 μm, the clearing reagent could quickly penetrate into the brain slices, and all brain slices became transparent in about 10 minutes. However, brain slices treated with different delipidation solutions showed different clearing and deformation processes. Brain slices treated with CUBIC and CUBIC-L delipidation solutions first experienced different degrees of contraction and then began to swell, while the brain slices treated with the delipidation solution S1 of the present invention showed a gentle expansion process during the clearing process. In addition, according to D of FIG. 4, after reaching the equilibrium state, brain slices treated with different clearing methods expanded in area by 46% (CUBIC), 30% (CUBIC-L) and 50% (S1 of the present invention), respectively.

The different deformation processes of the brain slices cleared with different clearing methods indicate that the clearing process of a biological tissue is affected by multiple interactions between the components of the biological tissue and the external chemical environment of the biological tissue, and the final deformation of the cleared biological tissue is the result of these interactions. The experimental results also indicate that the deformation caused by the clearing of biological tissues may be slightly anisotropic, which may be caused by a variety of reasons. First of all, the composition and chemical environment of different local areas of biological tissue are different, during the clearing process of tissue, as various physical and chemical reactions proceed and the clearing agents penetrate into the biological tissue, the composition and chemical environment of the biological tissue would change, thus producing uneven strains and stresses in biological tissues, causing anisotropic deformation of biological tissues. Secondly, the irregular physical contours and uneven mechanical properties of biological tissues may also lead to various degrees of deformation during the clearing process of biological tissues. Thirdly, fixed biological tissues are generally inelastic, resulting in the biological tissue being unable to return to its original shape after deformation. Therefore, during the clearing process of biological tissues, rapid expansion or contraction of biological tissue should be avoided as much as possible, so as to reduce the strain and stress that causes the deformation of biological tissue and maintain the integrity of biological tissues. The experimental results show that the delipidation reagent according to the present invention enables a smoother clearing process of biological samples, which is better for maintaining the integrity of biological tissues during the clearing process.

### Example 5 Clearing of 100 μm Thick Thy1-eGFP Mouse Brain Slices

In order to further study the impact of the delipidation reagent of the present invention on biological tissue structures, 100 μm thick Thy1-eGFP mouse brain slices were imaged before (in PBS solution) and after delipoidation with the delipidation solution S1 of the present invention and compared for their three-dimensional structural changes. The specific operations were as follows.

100 μm thick brain slices were cut from a fixed Thy1-eGFP adult mouse brain. In each experiment, a brain slice was placed in a glass bottom dish with a small amount of PBS solution added. The brain slice was covered with a coverslip and imaged with 20× and 63× objective lenses on a Zeiss LSM800 laser confocal microscope to obtain cellular and subcellular structural images of the brain slice. The coverslip was removed after imaging. The PBS solution was discarded and 2 ml of the delipidation solution S1 was added. The sample was let stand at 23° C. overnight. The excess S1 was discarded the next day. The brain slice was covered with a coverslip and imaged again using the Zeiss LSM800 laser confocal microscope to record the cellular and subcellular structural images of the delipidated brain slices.

FIG. 5 shows the changes in cell distribution and morphology of the Thy1-GFP-M mouse brain slices before and after delipidation using the delipidation solution S1 of the present invention, wherein, A shows the changes in cell distribution in the amygdala area before and after the delipidation, and the scale bar is 100 μm; and B shows the changes in cell morphology of a single cortex neuron before and after delipidation. The scale bars are (a, d) 30 μm, (b, e) 5 μm, (c, f) 0.5 μm.

In A of FIG. 5, the changes in cell distribution of mouse brain slices before and after clearing with the delipidation reagent of the present invention are compared at the cellular level resolution. The results show that the brain slices cleared using the delipidation solution S1 of the present invention has a high transparency, and more cells can be observed for the cleared mouse brain slices. The changes in the relative positions of the same cells before and after clearing the mouse brain slices indicates that the cleared mouse brain slices underwent expansion and slight anisotropic three-dimensional structural changes. However, since the anisotropic structural changes that occurred are very slight, it can be considered that mouse brain slices maintained approximately the same three-dimensional structural characteristics at the cellular level after cleared with the delipidation reagent of the present invention.

In B of FIG. 5, the morphological changes of a single neuron in the mouse brain slice before and after clearing with the delipidation reagent of the present invention are compared at subcellular level resolution. The results show that neuronal cells maintained almost the same dendritic and synaptic characteristics before and after clearing with the delipidation reagent of the present invention.

Therefore, the experimental results show that the structural characteristics of biological tissues can be well maintained at cellular to sub-cellular levels using the delipidation reagent of the present invention.

### Example 6 Imaging of w a Whole Adult Thy1-eGFP Mouse Brain

In order to test the performance of the delipidation reagent of the present invention to retain endogenous fluorescent proteins in biological tissue samples and the transparency of the cleared biological tissue samples, a tiling light sheet microscope was used to perform three dimensional imaging at different spatial resolutions on a whole adult Thy1-eGFP mouse brain cleared using the delipidation solution S1 of the present invention under different imaging conditions.

The clearing process of the whole adult Thy1-eGFP mouse brain was the same as that in Example 1.

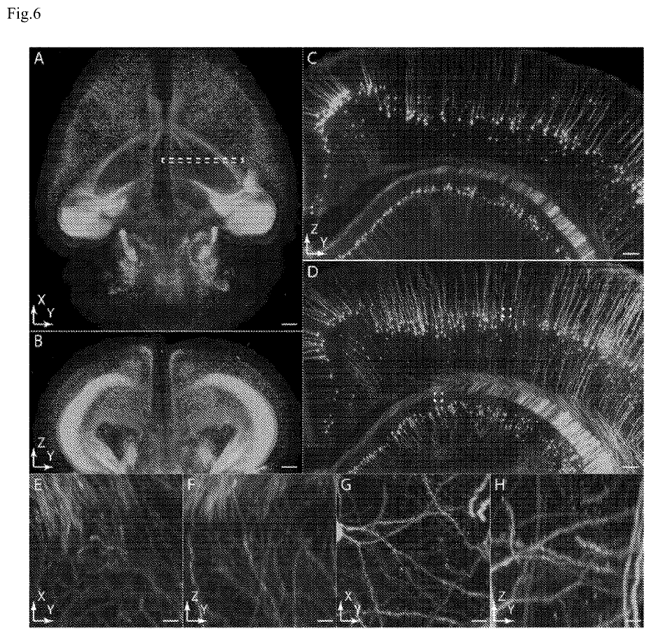

FIG. 6 shows the three-dimensional imaging of the whole mouse brain cleared with the delipidation solution S1 of the present invention at spatial resolutions from micron to sub-micron levels, wherein A and B are lateral and axial projection views of the three-dimensional imaging results of the cleared whole Thy1-eGFP mouse brain using a 0.25 numerical aperture (NA) air detection objective lens at a spatial resolution of 2×2×5 μm³, the scale bar is 1 mm; C is the axial projection view of the sample area marked in A, the scale bar is 200 μm; D is the axial projection view of the three-dimensional imaging results obtained by imaging the same area in C using a 0.5 NA immersion detection objective lens with a refractive index matching of 1.49 at a spatial resolution of 0.6×0.6×1.5 μm³, the scale bar is 200 μm; E-H are lateral and axial projection views of the two 100×100×100 μm³ regions marked in D, the scale bar is 10 μm.

The imaging results in A-C of FIG. 6 show the three-dimensional structure of the mouse brain neural network labeled by endogenous green fluorescent proteins. The spatial position and distribution of neurons in the labeled neural network can be clearly observed.

The imaging results in D-H of FIG. 6 show the subcellular morphology of neurons at sub-micron resolution, including features like neuronal dendrites, axons and synapses, as well as connections and projections between neurons. As shown in the imaging results, the mouse brain sample cleared using the delipidation reagent of the present invention has good transparency and retains endogenous fluorescent proteins of the sample efficiently, thereby allowing high-resolution three-dimensional imaging of the cleared mouse brain.

The present invention has been described in detail above. The delipidation reagent of the present invention has good clearing efficiency, clearing ability for large-volume samples, and retaining ability for endogenous fluorescent proteins. Meanwhile, the clearing process of biological samples is milder, so that it can better preserve the three-dimensional structural characteristics of biological tissues at cellular to subcellular levels and the integrity of biological tissues.

### REFERENCES

1. Li, A., et al., Micro-optical sectioning tomography to obtain a high-resolution atlas of the mouse brain. Science, 2010. 330 (6009): p. 1404-8.
2. Zhang, B., et al., Modified Golgi-Cox method for micrometer scale sectioning of the whole mouse brain. J Neurosci Methods, 2011. 197 (1): p. 1-5.
3. Maye, A., T. H. Wenckebach, and H. C. Hege, Visualization, reconstruction, and integration of neuronal structures in digital brain atlases. Int J Neurosci, 2006. 116 (4): p. 431-59.
4. Economo, M. N., et al., A platform for brain-wide imaging and reconstruction of individual neurons. Elife, 2016. 5: p. e10566.
5. Ragan, T., et al., Serial two-photon tomography for automated ex vivo mouse brain imaging. Nat Methods, 2012. 9 (3): p. 255-8.
6. Gong, H., et al., Continuously tracing brain-wide long-distance axonal projections in mice at a one-micron voxel resolution. Neuroimage, 2013. 74: p. 87-98.
7. Erturk, A., et al., Three-dimensional imaging of solvent-cleared organs using 3DISCO. Nat Protoc, 2012. 7 (11): p. 1983-95.
8. Gong, H., et al., High-throughput dual-colour precision imaging for brain-wide connectome with cytoarchitectonic landmarks at the cellular level. Nat Commun, 2016. 7: p. 12142.
9. Zhou, C., et al., Continuous imaging of large-volume tissues with a machinable optical clearing method at subcellular resolution. Biomed Opt Express, 2020. 11 (12): p. 7132-7149.
10. Chen, D., et al., Mapping the Brain-Wide Cholinergic Neurons Projecting to Skeletal Muscle in Mice by High-Throughput Light Sheet Tomography. Neurosci Bull, 2021. 37 (2): p. 267-270.
11. Ueda, H. R., et al., Whole-Brain Profiling of Cells and Circuits in Mammals by Tissue Clearing and Light-Sheet Microscopy. Neuron, 2020. 106 (3): p. 369-387.
12. Mano, T., et al., Whole-Brain Analysis of Cells and Circuits by Tissue Clearing and Light-Sheet Microscopy. J Neurosci, 2018. 38 (44): p. 9330-9337.
13. Tian, T., Z. Yang, and X. Li, Tissue clearing technique: Recent progress and biomedical applications. J Anat, 2021. 238 (2): p. 489-507.
14. Jing, D., et al., Tissue Clearing and Its Application to Bone and Dental Tissues. J Dent Res, 2019. 98 (6): p. 621-631.
15. Ueda, H. R., et al., Tissue clearing and its applications in neuroscience. Nat Rev Neurosci, 2020. 21 (2): p. 61-79.
16. Molbay, M., et al., A guidebook for DISCO tissue clearing. Mol Syst Biol, 2021. 17 (3): p. e9807.
17. Dodt, H. U., et al., Ultramicroscopy: three-dimensional visualization of neuronal networks in the whole mouse brain. Nat Methods, 2007. 4 (4): p. 331-6.
18. Pan, C., et al., Shrinkage-mediated imaging of entire organs and organisms using uDISCO. Nat Methods, 2016. 13 (10): p. 859-67.
19. Renier, N., et al., iDISCO: a simple, rapid method to immunolabel large tissue samples for volume imaging. Cell, 2014. 159 (4): p. 896-910.
20. Qi, Y., et al., FDISCO: Advanced solvent-based clearing method for imaging whole organs. Sci Adv, 2019. 5 (1): p. eaau8355.
21. Jing, D., et al., Tissue clearing of both hard and soft tissue organs with the PEGASOS method. Cell Res, 2018. 28 (8): p. 803-818.
22. Susaki, E. A., et al., Advanced CUBIC protocols for whole-brain and whole-body clearing and imaging. Nat Protoc, 2015. 10 (11): p. 1709-27.
23. Tainaka, K., et al., Chemical Landscape for Tissue Clearing Based on Hydrophilic Reagents. Cell Rep, 2018. 24 (8): p. 2196-2210 e9.
24. Murakami, T. C., et al., A three-dimensional single-cell-resolution whole-brain atlas using CUBIC-X expansion microscopy and tissue clearing. Nat Neurosci, 2018. 21 (4): p. 625-637.
25. Hama, H., et al., Scale: a chemical approach for fluorescence imaging and reconstruction of transparent mouse brain. Nat Neurosci, 2011. 14 (11): p. 1481-8.
26. Zhu, X., et al., Ultrafast optical clearing method for three-dimensional imaging with cellular resolution. Proc Natl Acad Sci USA, 2019. 116 (23): p. 11480-11489.
27. Susaki, E. A., et al., Versatile whole-organ/body staining and imaging based on electrolyte-gel properties of biological tissues. Nat Commun, 2020. 11 (1): p. 1982.
28. Gradinaru, V., et al., Hydrogel-Tissue Chemistry: Principles and Applications. Annu Rev Biophys, 2018. 47: p. 355-376.
29. Tomer, R., et al., Advanced CLARITY for rapid and high-resolution imaging of intact tissues. Nat Protoc, 2014. 9 (7): p. 1682-97.
30. Epp, J. R., et al., Optimization of CLARITY for Clearing Whole-Brain and Other Intact Organs. eNeuro, 2015. 2 (3).
31. Du, H., et al., Advances in CLARITY-based tissue clearing and imaging. Exp Ther Med, 2018. 16 (3): p. 1567-1576.
32. Du, H., et al., Modified CLARITY Achieving Faster and Better Intact Mouse Brain Clearing and Immunostaining. Sci Rep, 2019. 9 (1): p. 10571.
33. Greenbaum, A., et al., Bone CLARITY: Clearing, imaging, and computational analysis of osteoprogenitors within intact bone marrow. Sci Transl Med, 2017. 9 (387).
34. Ku, T., et al., Multiplexed and scalable super-resolution imaging of three-dimensional protein localization in size-adjustable tissues. Nat Biotechnol, 2016. 34 (9): p. 973-81.
35. Yang, B., et al., Single-cell phenotyping within transparent intact tissue through whole-body clearing. Cell, 2014. 158 (4): p. 945-958.
36. Kim, S. Y., et al., Stochastic electrotransport selectively enhances the transport of highly electromobile molecules. Proc Natl Acad Sci USA, 2015. 112 (46): p. E6274-83.
37. Park, Y. G., et al., Protection of tissue physicochemical properties using polyfunctional crosslinkers. Nat Biotechnol, 2018.
38. Truckenbrodt, S., et al., A practical guide to optimization in X10 expansion microscopy. Nat Protoc, 2019. 14 (3): p. 832-863.

1. A delipidation composition comprising urea, N-butyldiethanolamine and Triton X-100, wherein the mass ratio of urea, N-butyldiethanolamine and Triton-100 is (0.6-5):(0.3-3): 1.

2. The delipidation composition according to claim 1, wherein the mass ratio of urea, N-butyldiethanolamine and triton X-100 is (1-2.25):(0.6-1.5): 1, preferably 1.5:1:1.

3. Use of the delipidation composition of claim 1 or 2 for preparing a delipidation reagent.

4. A delipidation reagent, which is an aqueous solution containing, by mass percentage concentration, 10 to 25% of urea, 5 to 15% of N-butyldiethanolamine and 5 to 15% of Triton X-100.

5. The delipidation reagent according to claim 5, wherein,
the mass percentage of urea is 12 to 18%, preferably 15%;
The mass percentage of N-butyldiethanolamine is 8 to 12%, preferably 10%;
The mass percentage of Triton X-100 is 8 to 12%, preferably 10%.

6. A kit for clearing and imaging of biological tissues, which comprises the delipidation composition of claim 1 or 2 or the delipidation reagent of claim 4 or 5.

7. The kit according to claim 6, further comprising one or more selected from the group consisting of a phosphate buffer, paraformaldehyde, a refractive index matching composition, a gelling reagent, an imaging buffer, and a staining reagent.

8. The kit according to claim 7, wherein,
the phosphate buffer is a phosphate buffer composed of disodium hydrogen phosphate and sodium dihydrogen phosphate with a pH of 7.0 to 7.4, preferably 7.2;
the paraformaldehyde is a solid, or a 4% paraformaldehyde solution by g/ml;
the refractive index matching composition is a solid composed of a refractive index matching reagent from which the prepared refractive index matching solution has a refractive index of 1.46 to 1.50, or a refractive index matching solution with a refractive index of 1.46 to 1.50;
the gelling reagent is an agarose solid, or an agarose solution with a mass percentage concentration of 1.5% to 3%, preferably 1.8% to 2.5%, especially 2%;
the imaging buffer is a mixture of silicone oil and mineral oil with a refractive index which is 99.8% to 100.06%, preferably 99.9% to 100.01% of the refractive index of the refractive index matching solution, in particular, the imaging buffer is a mixture of silicone oil and mineral oil with a refractive index of 1.494.

9. The kit according to claim 7, wherein,
the refractive index matching composition contains urea, sucrose, antipyrine and trihydroxyethylamine, wherein the ratio of urea, sucrose, antipyrine and trihydroxyethylamine is (1-7):(1-6.5):(1-6.5): 1, especially (1.6-3.8):(1.6-3.2):(1.6-3.2): 1, more especially 2.5:2.25:2.25:1;
in particular, the refractive index matching composition is an aqueous solution, which contains, by mass percentage concentration, 15 to 35%, preferably 20 to 30%, especially 25% of urea, 15 to 32.5%, preferably 20 to 25%, especially 22.5% of sucrose, 15 to 32.5%, preferably 20 to 25%, especially 22.5% of antipyrine, and 5 to 15%, preferably 8 to 12%, especially 10% of trihydroxyethylamine.

10. The kit according to claim 7, wherein the kit includes:
(1) a fast delipidation solution, which is an aqueous solution containing by mass percentage concentration, 15% of urea, 10% of N-butyldiethanolamine and 10% of Triton X-100;
(2) a refractive index matching solution, which is an aqueous solution containing, by mass percentage concentration, 25% of urea, 22.5% of sucrose, 22.5% of antipyrine and 10% of trihydroxyethylamine;
(3) a gelling solution, which is a solution of 2% by mass percentage concentration of agarose in the refractive index matching solution;
(4) an imaging buffer, which is a mixture of silicone oil and mineral oil with a refractive index of 1.494.

11. A method for clearing a biological tissue, which comprises: delipidating a biological tissue sample in a delipidation reagent of claim 4 or 5 until the sample is transparent, and then matching the refractive index of the biological tissue sample in a refractive index matching solution.

12. The method of claim 11, wherein,
the delipidation reagent is replaced regularly or irregularly, and the amount of the delipidation reagent at single use is 5 to 25 times, preferably 10 to 20 times, especially 15 times of the volume of the sample;
the refractive index matching solution is an aqueous solution, which contains, by mass percentage concentration, 15 to 35%, preferably 20 to 30%, especially 25% of urea, 15 to 32.5%, preferably 20 to 25%, especially 22.5% of sucrose, 15 to 32.5%, preferably 20 to 25%, especially 22.5% of antipyrine, and 5 to 15%, preferably 8 to 12%, especially 10% of trihydroxyethylamine,
the refractive index matching solution is replaced regularly or irregularly, and the amount of the refractive index matching solution at single use is 2 to 22 times, preferably 7 to 17 times, especially 12 times of the volume of the sample.

13. A biological tissue clearing and imaging method comprising a step of clearing the biological tissue sample using the method of claim 11 or 12.

14. The imaging method according to claim 13, comprising steps of:
(1) fixing a biological tissue sample;
(2) clearing the fixed biological tissue sample with the method of claim 11 or 12;
(3) gel-embedding the cleared biological tissue sample;
(4) imaging the gel-embedded biological tissue sample.
